(19) United States
(12) Patent Application Publication
(10) Pub. No.: US 2018/0214462 A1
(43) Pub. Date: Aug. 2, 2018

(54) BLOOD-BRAIN-BARRIER PERMEABILITY MODULATORS AND USES THEREOF

(71) Applicant: B.G. NEGEV TECHNOLOGIES AND APPLICATIONS LTD. AT BEN-GURION UNIVERSITY, Beer Sheva (IL)

(72) Inventors: Alon FRIEDMAN, Mosherville (CA); Yehuda VAZANA, Ashdod (IL)

(21) Appl. No.: 15/747,646

(22) PCT Filed: Jul. 27, 2016
(86) PCT No.: PCT/IL2016/050821

§ 371 Date: Jan. 25, 2018

Related U.S. Application Data
(60) Provisional application No. 62/200,165, filed on Aug. 3, 2015.

(51) Int. Cl. A61K 31/662; A61P 25/00; A61K 31/675

(52) U.S. Cl. CPC A61K 31/662; A61K 31/675; A61P 25/00

(57) ABSTRACT

Provided are compositions which include N-methyl-d-aspartate receptor (NMDA-R) antagonists. Moreover, provided are methods for reducing the permeability of the blood-brain-barrier, in a patient, by administering to the patient, a composition which includes N-methyl-d-aspartate receptor (NMDA-R) antagonists.

### FIELD OF INVENTION

This invention is directed to; inter alia, to composition and methods for modulating the permeability of the blood-brain-barrier.

### BACKGROUND OF THE INVENTION

The blood-brain-barrier (BBB) is a highly specialized interface that separates the circulating blood from the brain's extracellular fluid in the CNS. The BBB is formed at the level of endothelial cells which are connected by tight-junction protein complexes that seal together the para-cellular space. It consists of specialized transcellular transport systems, a basal membrane and astrocytic end-feet (Abbott et al., 2006). The selective nature of the BBB allows the formation of a unique extracellular milieu within brain neuropil (Abbott et al., 2006), essential for normal brain function. In most common brain disorders, including epilepsy, traumatic brain injury, stroke and neurodegenerative diseases, the BBB may be compromised (Benveniste et al., 1984; Brown and Davis, 2002; Davies, 2002; Friedman, 2011; Nishizawa, 2001; Seiffert et al., 2004; Van Vliet et al., 2007) and could contribute to neural dysfunction, neural network reorganization and degeneration, thus modifying disease progression (Benveniste et al., 1984; Seiffert et al., 2004; Tomkins et al., 2008; Van Vliet et al., 2007). However, the mechanisms underlying BBB dysfunction in brain disorders are not fully understood.

The potential of excessive neuronal activation to increase brain vascular permeability to blood constituents is supported by the following indirect evidence: (1) Seizures, and in particular when recurrent or prolonged, such as in status epilepticus, are associated with BBB dysfunction (Friedman, 2011; Nitsch and Hubauer, 1986); (2) Increased BBB permeability is often a hallmark of the perilesional brain in ischemia, trauma and tumors—neurological conditions associated with neuronal hyper-excitability, epileptic seizures and spreading depolarizations (Davies, 2002; Schoknecht et al., 2014; Tomkins et al., 2008); (3) The major excitatory neurotransmitter, glutamate, has been demonstrated to increase permeability in cultured brain endothelial cells (András et al., 2007; Sharp et al., 2003); and (4) Whole brain stimulation, such as that performed during electro-convulsive treatment for severe depression, has been shown to accompany increased glutamate levels (Zangen and Hyodo, 2002) and BBB breakdown (Mottaghy et al., 2003).

### SUMMARY OF THE INVENTION

In one embodiment, provided herein is a method for reducing the permeability of the blood-brain-barrier, in a subject in need thereof, comprising administering to the subject, a composition comprising N-methyl-d-aspartate receptor (NMDA-R) antagonist, thereby reducing the permeability of the BBB, in a subject in need thereof. In one embodiment, the NMDA-R antagonist is a competitive antagonist. In one embodiment, the NMDA-R antagonist is APV, R-2-amino-5-phosphonopentanoate (AP5), memantine, 2-amino-7-phosphonoheptanoic acid (AP7), 3-[(R)-2-carboxypiperazin-4-yl]-prop-2-enyl-1-phosphonic acid (CPPene), (2S,4R)-4-(phosphonomethyl)piperidine-2-carboxylic acid (Selfotel), Dexanabinol (HU-211), (+)-5-methyl-10,11-dihydro-5H-dibenzo[a,d]cyclohepten-5,10-imine (MK-801), (+−)-6-phosphonomethyl-decahydroisoquinolin-3-carboxylic acid (LY274614), 2-amino-5-phosphonovalerate (AF5), (cis-2-carboxypiperidine-4-yl)-methyl-1-phosphonic acid (CGS19755 1), cis-(±)-4-(2H-tetrazol-5-yl)methylpiperidine-2-carboxylicacid (LY233053), 2-amino-4; 5-(1; 2-cyclohexyl)-7-phosphonoheptanoic acid (NPC12626), or any combination thereof.

In one embodiment, a subject to be treated with the compositions and methods as described herein is afflicted with brain seizures. In one embodiment, a subject to be treated with the compositions and methods as described herein is afflicted with status epilepticus or recurrent seizures that induce BBB opening that may facilitate brain injury. In the attached graph you can see that NMDA-antagonist given to an animal during recurrent seizures (by a chemical convalescent 4-AP) was efficient in reducing BBB breakdown while it did not block the seizures. In one embodiment, a subject to be treated with the compositions and methods as described herein is afflicted with a brain injury. In one embodiment, a subject to be treated with the compositions and methods as described herein is afflicted with hyper BBB permeability. In one embodiment, a subject to be treated with the compositions and methods as described herein is afflicted with brain ischemia.

In one embodiment, further provided herein is a method for preventing an increase in the permeability of the BBB, in a subject in need thereof, comprising administering to the subject, a composition comprising N-methyl-d-aspartate receptor (NMDA-R) antagonist, thereby preventing an increase in the permeability of the blood-brain-barrier, in a subject in need thereof.

### BRIEF DESCRIPTION OF THE DRAWINGS

FIGS. 1A-G: BBB dysfunction following focal cortical seizures and excess glutamate. A. Top: ECoG recordings from an anesthetized rat prior to (ACSF) and post topical 4AP application (+4AP). Following 60′ under 4AP exposure, recurrent seizures were recorded. Bottom: seizure activity was accompanied by an increase in vessel diameter (10.05±1.01%, n=8, p=0.01). B. Fluorescence imaging prior to (ACSF) and 70′ following 4AP (+4AP 70′) showing extra-vascular dye, indicative of BBB dysfunction. C. The effect of recurrent seizures on vessels' permeability is noticed by EB (Evans blue, see Methods) extravasation in the treated hemisphere alone. D. BBB permeability maps (color codes for the extent of permeability, see Materials and Methods) depicting the effect of recurrent seizures. E. Averaged change in BBB permeability (see Materials and Methods) during seizures. F. Permeability maps in response to the application of glutamate for 30′ (+glut 1 mM, 30′).G. Dose response showing a gradual change in vessels' permeability due to increased concentrations of glutamate (1 mM, p=0.02, n=9). * p<0.05.

FIGS. 2A-D: Mechanisms underlying glutamate-induced BBB opening. A. Change in vessels' permeability under different experimental conditions: cortical glutamate application (1 mM, glut), NMDA application (1 mM, NMDA), the addition of D-AP5 (0.05 mM, glut+D-AP5) and ACSF exposure only (control). B. Probe-based confocal laser endomicroscopy (PCLE, Cellvizio Dual Band, Mauna Kea Tech.) of a rat neocortex, following intravenous injection of Evans Blue (EB, see Methods) and topical application of the calcium indicator, Oregon green-BAPTA-1AM (BOG, see Methods). Calcium signal was assessed in the vessel lumen (white dashed line). C-D. Rise in calcium signal is shown in the vessel wall following the drop application of glutamate; it was found to be significant. * p<0.05 **p<0.01

FIGS. 3A-C: Blood-brain barrier disruption in the peri-ischemic cortex is prevented by the NMDA receptor antagonist D-AP5. A. The development of a photothrombosis (PT, see Materials and Methods) as imaged using intravital microscopy after the injection of Na Fluorescein (red arrow indicates damaged vasculature). Increased vessels' permeability (Na Fluorescein leakage) is observed in the peri-ischmic cortex at 60 minutes follow-up (PT 60′, ACSF). B. The peri-infarct. Perfused cortex with abnormal permeability is color coded. C. Averaged percent increase in permeability in the peri-infarct cortex at 30 and 60 min following PT shows a marked increase in permeability in control (ACSF) rats (n=9, p=0.008, Wilcoxon) compared to a non-significant change in animals exposed to PT in the presence of the NMDA-R antagonist, D-AP5 (n=7 p=0.13, Wilcoxon). * p<0.05 **p<0.01.

FIGS. 4A-F: Direct cortical imaging in anesthetized rodents is analyzed for quantitative assessment of BBB permeability. A. A rat is anesthetized and placed in a stereotactic frame (see Methods). B. Craniotomy is used to expose a neocortical section (see Methods). C. Na Fluorescein (NaFlu) is applied IV. Vessel interior in the exposed section is illuminated. D. Rescaling and segmentation of the fluorescence image. The product is a binary image in which vascular and extra-vascular areas are contrasted. E. Averaging pixel intensities through time in the primary artery (marked by red frame in c.) forms the arterial input function (AIF, primary artery, red). Each extra-vascular pixel is also represented by an intensity-time (IT) curve (extra-vascular, black). Tracer residues in extra-vascular space are assessed by comparing both functions in the marked time span (arrow). The result is a per-pixel numerical parameter reflective of BBB permeability level (permeability index-PI). F. Spatial mapping of BBB permeability.

FIGS. 5A-E: BBB permeability assessment with DCE-MRI analysis in human subjects. A. T1-weighted MRI scan of a patient following tumor resection. B. 1st and 7th dynamic scans. C. Normalized slope map indicating BBB permeability. D. Cumulative distribution functions (CDF) of the normalized slope values of 4 control subjects (whole brain, green) and 10 patients following tumor resection in the contralateral hemisphere to the tumor (blue) and the tumor bed (TB, red). The total CDF of controls+contralateral hemisphere to the tumor is in black. Dashed lines indicate the global threshold value of 0.0109, derived from the 95th percentile of the total CDF. E. Voxels with supra-threshold (ST) slope values.

FIG. 6A-D: NMDA receptor antagonists diminish BBB breakdown following seizures: A. ECoG recordings in the exposed rat neocortex. Following 4AP application, a rise in amplitude and frequency is observed indicating seizure activity. B. Color-coded maps exhibit a rise in vessel permeability immediately (<10 min) following the seizure (4AP) in comparison to normal neuronal activity (ACSF). C. Seizure-induced permeability increase is diminished (p=0.02, Mann-Whitney) when NMDA receptor antagonists (NMDAR-A, Memantine 40 mg/kg IP/D-AP5 100 μM topical) are applied. D. Quantification of vascular features and seizure activity. The topical application of glutamate (1 mM) in combination with axonal and synaptic transmission blockers (Glut+blockers) generated a similar increase in vessel permeability as seen under seizure activity while not inducing a shift in vascular diameter. * p<0.05

### DETAILED DESCRIPTION OF THE INVENTION

In one embodiment, provided herein a method for modulating the permeability of the blood-brain-barrier, in a subject in need thereof. In one embodiment, provided herein a method for treating a BBB pathology, in a subject in need thereof. In one embodiment, provided herein a method for reducing the permeability of the blood-brain-barrier, in a subject in need thereof. In one embodiment, provided herein a method for enhancing and/or increasing the permeability of the blood-brain-barrier, in a subject in need thereof. In one embodiment, a method for modulating (both: reducing/inhibiting or increasing/enhancing) the permeability of the blood-brain-barrier is reversible.

In one embodiment, NMDAR antagonists as described herein are used for facilitating BBB closure. In one embodiment, NMDAR antagonists as described herein induce and/or enhance BBB closure. In one embodiment, NMDAR antagonists as described herein decrease BBB permeability. In one embodiment, NMDAR antagonists as described herein decrease the rate of flow through the BBB. In one embodiment, NMDAR antagonists of the present invention transiently and/or reversibly modify BBB permeability, closure.

In one embodiment, NMDAR antagonists of the present invention treat, inhibit, reduce the risk, reduce harmful effects, and/or ameliorate pathologies, including: stroke, Alzheimer's disease, non-Alzheinmr's nerurodegenerative diseases, acute liver failure, multiple sclerosis, meningitis, HIV, diabetes, depressive and psychotic disorders, cerebral malaria, Parkinson's disease, traumatic and surgical brain injury, concussion, peripheral nerve injury, brain cancer, epilepsy and peripheral inflammatory pain.

In one embodiment, a method for modulating (both: reducing/inhibiting or increasing/enhancing) the permeability of the blood-brain-barrier is reversible within 30 minutes to 24 hours. In one embodiment, a method for modulating (both: reducing/inhibiting or increasing/enhancing) the permeability of the blood-brain-barrier is reversible within 30 minutes to 14 hours. In one embodiment, a method for modulating (both: reducing/inhibiting or increasing/enhancing) the permeability of the blood-brain-barrier is reversible within 30 minutes to 10 hours. In one embodiment, a method for modulating (both: reducing/inhibiting or increasing/enhancing) the permeability of the blood-brain-barrier is reversible within 30 minutes to 5 hours. In one embodiment, a method for modulating (both: reducing/inhibiting or increasing/enhancing) the permeability of the blood-brain-barrier is reversible within 1 hour to 15 hours. In one embodiment, a method for modulating (both: reducing/inhibiting or increasing/enhancing) the permeability of the blood-brain-barrier is reversible within 2 hours to 10 hours. In one embodiment, a method for modulating (both: reducing/inhibiting or increasing/enhancing) the permeability of the blood-brain-barrier is reversible within 2 hours to 6 hours.

In one embodiment, provided herein a method for reducing the permeability of the blood-brain-barrier or treating a BBB pathology, in a subject in need thereof, comprising administering to the subject, a composition comprising N-methyl-d-aspartate receptor (NMDA-R) antagonist, thereby reducing the permeability of the blood-brain-barrier or treating a BBB pathology, in a subject in need thereof. In one embodiment, NMDA receptor antagonist is known as an anesthetic. In one embodiment, NMDA receptor antagonist inhibits the action of, the N-Methyl-D-aspartate receptor (NMDAR). In one embodiment, NMDA receptor antagonist is an opioid.

In one embodiment, NMDA receptor antagonist is an uncompetitive channel blocker. In one embodiment, NMDA receptor antagonist is Amantadine or amantadine sulfate. In one embodiment, NMDA receptor antagonist is Atomoxetine. In one embodiment, NMDA receptor antagonist is AZD6765. In one embodiment, NMDA receptor antagonist is Chloroform. In one embodiment, NMDA receptor antagonist is Dextrallorphan. In one embodiment, NMDA receptor antagonist is Dextromethorphan. In one embodiment, NMDA receptor antagonist is Dextrorphan. In one embodiment, NMDA receptor antagonist is Diphenidine. In one embodiment, NMDA receptor antagonist is Dizocilpine (MK-801). In one embodiment, NMDA receptor antagonist is Eticyclidine. In one embodiment, NMDA receptor antagonist is Ethanol. In one embodiment, NMDA receptor antagonist is Gacyclidine. In one embodiment, NMDA receptor antagonist is Ibogaine. In one embodiment, NMDA receptor antagonist is Magnesium. In one embodiment, NMDA receptor antagonist is Memantine. In one embodiment, NMDA receptor antagonist is Methoxetamine. In one embodiment, NMDA receptor antagonist is Nitromemantine. In one embodiment, NMDA receptor antagonist is Phencyclidine. In one embodiment, NMDA receptor antagonist is Rolicyclidine. In one embodiment, NMDA receptor antagonist is Tenocyclidine. In one embodiment, NMDA receptor antagonist is Methoxydine. In one embodiment, NMDA receptor antagonist is Tiletamine. In one embodiment, NMDA receptor antagonist is Xenon. In one embodiment, NMDA receptor antagonist is Neramexane. In one embodiment, NMDA receptor antagonist is Eliprodil. In one embodiment, NMDA receptor antagonist is Etoxadrol. In one embodiment, NMDA receptor antagonist is Dexoxadrol. In one embodiment, NMDA receptor antagonist is WMS-2539. In one embodiment, NMDA receptor antagonist is Remacemide. In one embodiment, NMDA receptor antagonist is NEFA. In one embodiment, NMDA receptor antagonist is Delucemine. In one embodiment, NMDA receptor antagonist is 8 A-PDHQ.

In one embodiment, NMDA receptor antagonist is a synthetic opioid. In one embodiment, NMDA receptor antagonist is pethidine. In one embodiment, NMDA receptor antagonist is methadone. In one embodiment, NMDA receptor antagonist is dextropropoxyphene. In one embodiment, NMDA receptor antagonist is tramadol. In one embodiment, NMDA receptor antagonist is ketobemidone.

In one embodiment, NMDA receptor antagonist is ketamine. In one embodiment, NMDA receptor antagonist is dextromethorphan (DXM). In one embodiment, NMDA receptor antagonist is phencyclidine (PCP). In one embodiment, NMDA receptor antagonist is Methoxetamine (MXE). In one embodiment, NMDA receptor antagonist is nitrous oxide (N₂O).

In one embodiment, NMDA receptor antagonist is a competitive antagonist. In one embodiment, NMDA receptor antagonist is AP5(APV, R-2-amino-5-phosphonopentanoate). In one embodiment, NMDA receptor antagonist is AP7 (2-amino-7-phosphonoheptanoic acid). In one embodiment, NMDA receptor antagonist is CPPene (3-[(R)-2-carboxypiperazin-4-yl]-prop-2-enyl-1-phosphonic acid). In one embodiment, NMDA receptor antagonist is Selfotel.

In one embodiment, NMDA receptor antagonist is a non-competitive antagonist. In one embodiment, NMDA receptor antagonist is Aptiganel (Cerestat, CNS-1102). In one embodiment, NMDA receptor antagonist is HU-211. In one embodiment, NMDA receptor antagonist is Remacemide. In one embodiment, NMDA receptor antagonist is Rhynchophylline. In one embodiment, NMDA receptor antagonist is Ketamine.

In one embodiment, NMDA receptor antagonist is a Glycine antagonist. In one embodiment, NMDA receptor antagonist is Rapastinel (GLYX-13). In one embodiment, NMDA receptor antagonist is NRX-1074. In one embodiment, NMDA receptor antagonist is -7Chlorokynurenic acid. In one embodiment, NMDA receptor antagonist is 4-Chlorokynurenine (AV-101). In one embodiment, NMDA receptor antagonist is 5,7-Dichlorokynurenic acid. In one embodiment, NMDA receptor antagonist is Kynurenic acid. In one embodiment, NMDA receptor antagonist is TK-40. In one embodiment, NMDA receptor antagonist is 1-Aminocyclopropanecarboxylic acid. In one embodiment, NMDA receptor antagonist is L-Phenylalanine. In one embodiment, NMDA receptor antagonist comprises any NMDA receptor antagonist known in the art. In one embodiment, NMDA receptor antagonist comprises a combination of NMDA receptor antagonists.

In one embodiment, the NMDA-R antagonist is APV, R-2-amino-5-phosphonopentanoate (AP5), memantine, 2-amino-7-phosphonoheptanoic acid (AP7), 3-[(R)-2-carboxypiperazin-4-yl]-prop-2-enyl-1-phosphonic acid (CPPene), (2S,4R)-4-(phosphonomethyl)piperidine-2-carboxylic acid (Selfotel), Dexanabinol (HU-211), (+)-5-methyl-10,11-dihydro-5H-dibenzo[a,d]cyclohepten-5,10-imine (MK-801), (+−)-6-phosphonomethyl-decahydroisoquinolin-3-carboxylic acid (LY274614), 2-amino-5-phosphonovalerate (AF5), (cis-2-carboxypiperidine-4-yl)-methyl-1-phosphonic acid (CGS19755 1), cis-(±)-4-(2H-tetrazol-5-yl)methylpiperidine-2-carboxylicacid (LY233053), 2-amino-4; 5-(1; 2-cyclohexyl)-7-phosphonoheptanoic acid (NPC12626), or any combination thereof.

In one embodiment, a subject is a human subject. In one embodiment, a subject is an animal. In one embodiment, a subject is a farm animal. In one embodiment, a subject is a pet.

In one embodiment, a subject in need of a method for reducing and/or inhibiting BBB permeability suffers from brain seizures. In one embodiment, a subject in need of a method for reducing and/or inhibiting BBB permeability suffers from reoccurring brain seizures. In one embodiment, a subject in need of a method for reducing and/or inhibiting BBB permeability suffers from a brain trauma. In one embodiment, a subject in need of a method for reducing and/or inhibiting BBB permeability suffers from a brain injury. In one embodiment, a subject in need of a method for reducing and/or inhibiting BBB permeability suffers from brain ischemia.

In one embodiment, a subject in need of a method as described herein is afflicted with a BBB pathology. In one embodiment, a subject in need of a method for reducing and/or inhibiting BBB permeability suffers from Meningitis. In one embodiment, a subject in need of a method for reducing and/or inhibiting BBB permeability suffers from Brain abscess. In one embodiment, a subject in need of a method for reducing and/or inhibiting BBB permeability suffers from epilepsy. In one embodiment, a subject in need of a method for reducing and/or inhibiting BBB permeability suffers from multiple sclerosis. In one embodiment, a subject in need of a method for reducing and/or inhibiting BBB permeability suffers from Neuromyelitis optica. In one embodiment, a subject in need of a method for reducing and/or inhibiting BBB permeability suffers from Progressive multifocal leukoencephalopathy (PML). In one embodiment, a subject in need of a method for reducing and/or inhibiting BBB permeability suffers from Cerebral edema. In one embodiment, a subject in need of a method for reducing and/or inhibiting BBB permeability suffers from HIV encephalitis. In one embodiment, a subject in need of a method for reducing and/or inhibiting BBB permeability suffers from cerebral malaria. In one embodiment, a subject in need of a method for reducing and/or inhibiting BBB permeability suffers from or infected by Rabies.

In another embodiment, the present methods provide preventive measures for a subject susceptible of acquiring a brain disease or at risk of a brain disease deterioration. In one embodiment, a subject in need of preventive measures is in contact with patients afflicted with Meningitis. In one embodiment, a subject in need of preventive measures participates in athletic or sport activity that often results in brain injuries.

In one embodiment, a subject in need of preventive measures suffers from epilepsy. In one embodiment, a subject in need of preventive measures suffers from multiple sclerosis. In one embodiment, a subject in need of preventive measures has high risk for being infected with HIV. In one embodiment, a subject in need of preventive measures is exposed to Rabies. In one embodiment, a subject in need of preventive measures is at risk of a brain injury. In one embodiment, a subject in need of preventive measures is at risk of a traumatic brain injury (TBI).

In one embodiment, a subject in need of a method for enhancing and/or inducing BBB permeability suffers from De Vivo disease.

In one embodiment, a subject to be treated with the compositions and methods as described herein is afflicted with hyper BBB permeability. In one embodiment, a subject to be treated with the compositions and methods as described herein is afflicted with over-permeability of the BBB. In one embodiment, a subject to be treated with the compositions and methods as described herein is in need of BBB closure and/or reduction of BBB permeability.

In one embodiment, modulating BBB permeability is modulating the permeability of endothelial cells, which are connected by tight junctions. In one embodiment, modulating BBB permeability is modulating the BBB's electrical resistivity. In one embodiment, modulating BBB permeability is modulating the permeability of capillaries associated with the BBB. In one embodiment, modulating BBB permeability is modulating the permeability of the brain's capillary endothelium.

In one embodiment, provided herein a method for preventing an increase in the permeability of the blood-brain-barrier, in a subject in need thereof, comprising administering to the subject, a composition comprising N-methyl-d-aspartate receptor (NMDA-R) antagonist, thereby preventing an increase in the permeability of the blood-brain-barrier, in a subject in need thereof. In one embodiment, provided herein a method for reducing flow rate through the BBB, in a subject in need thereof, comprising administering to the subject, a composition comprising N-methyl-d-aspartate receptor (NMDA-R) antagonist. In one embodiment, a composition comprising NMDA-R antagonist is a composition comprising an effective amount of NMDA-R antagonist.

In one embodiment, administering is topically administering. In one embodiment, administering is orally administering. In one embodiment, administering is systemically administering. In one embodiment, administering is intravenously administering. In one embodiment, administering is intranasaly administering. In one embodiment, administering is intravenously administering. In one embodiment, administering is intramuscularly administering.

In one embodiment, modulating the permeability of the BBB is modulating the diameter of a blood vessel in the blood-brain-barrier. In one embodiment, reducing and/or increasing the permeability of the BBB is reducing and/or increasing flow rate through the BBB.

In one embodiment, reducing the permeability of the BBB is treating a BBB pathology.

In one embodiment, NMDA-R antagonist of the invention is provided or utilized within a pharmaceutical composition. In one embodiment, a “pharmaceutical composition” refers to a preparation of one or more of the NMDA-R antagonists described herein with other chemical components such as physiologically suitable carriers and excipients. The purpose of a pharmaceutical composition is to facilitate administration of a compound to an organism. In one embodiment, a “pharmaceutical composition” comprises a “physiologically acceptable carrier”.

In one embodiment, the phrases “physiologically acceptable carrier” and “pharmaceutically acceptable carrier” which be interchangeably used refer to a carrier or a diluent that does not cause significant irritation to an organism and does not abrogate the biological activity and properties of the administered compound. An adjuvant is included under these phrases. In one embodiment, one of the ingredients included in the pharmaceutically acceptable carrier can be for example polyethylene glycol (PEG), a biocompatible polymer with a wide range of solubility in both organic and aqueous media (Mutter et al. (1979).

In one embodiment, a “pharmaceutical composition” comprises an excipient. In one embodiment, “excipient” refers to an inert substance added to a pharmaceutical composition to further facilitate administration of a NMDA-R antagonists. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, Pa., latest edition, which is incorporated herein by reference.

In one embodiment, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In one embodiment, the preparation is administered in a local rather than systemic manner, for example, via injection of the preparation directly into a specific region of a patient's body.

Various embodiments of dosage ranges are contemplated by this invention. The dosage of the NMDA-R antagonists of the present invention, in one embodiment, is in the range of 0.05-80 mg/day. In another embodiment, the dosage is in the range of 0.05-50 mg/day. In another embodiment, the dosage is in the range of 0.1-20 mg/day. In another embodiment, the dosage is in the range of 0.1-10 mg/day. In another embodiment, the dosage is in the range of 0.1-5 mg/day. In another embodiment, the dosage is in the range of 0.5-5 mg/day. In another embodiment, the dosage is in the range of 0.5-50 mg/day. In another embodiment, the dosage is in the range of 5-80 mg/day. In another embodiment, the dosage is in the range of 35-65 mg/day. In another embodiment, the dosage is in the range of 35-65 mg/day. In another embodiment, the dosage is in the range of 20-60 mg/day. In another embodiment, the dosage is in the range of 40-60 mg/day. In another embodiment, the dosage is in a range of 45-60 mg/day. In another embodiment, the dosage is in the range of 40-60 mg/day. In another embodiment, the dosage is in a range of 60-120 mg/day. In another embodiment, the dosage is in the range of 120-240 mg/day. In another embodiment, the dosage is in the range of 40-60 mg/day. In another embodiment, the dosage is in a range of 240-400 mg/day. In another embodiment, the dosage is in a range of 45-60 mg/day. In another embodiment, the dosage is in the range of 15-25 mg/day. In another embodiment, the dosage is in the range of 5-10 mg/day. In another embodiment, the dosage is in the range of 55-65 mg/day.

In one embodiment, the dosage is 20 mg/day. In another embodiment, the dosage is 30 mg/day. In another embodiment, the dosage is 40 mg/day. In another embodiment, the dosage is 50 mg/day. In another embodiment, the dosage is 60 mg/day. In another embodiment, the dosage is 70 mg/day. In another embodiment, the dosage is 80 mg/day. In another embodiment, the dosage is 90 mg/day. In another embodiment, the dosage is 100 mg/day.

Oral administration, in one embodiment, comprises a unit dosage form comprising tablets, capsules, lozenges, chewable tablets, suspensions, emulsions and the like. Such unit dosage forms comprise a safe and effective amount of the desired compound, or compounds, each of which is in one embodiment, from about 0.7 or 3.5 mg to about 280 mg/70 kg, or in another embodiment, about 0.5 or 10 mg to about 210 mg/70 kg. The pharmaceutically-acceptable carriers suitable for the preparation of unit dosage forms for peroral administration are well-known in the art. In some embodiments, tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. In one embodiment, glidants such as silicon dioxide can be used to improve flow characteristics of the powder-mixture. In one embodiment, coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. In some embodiments, the selection of carrier components depends on secondary considerations like taste, cost, and shelf stability, which are not critical for the purposes of this invention, and can be readily made by a person skilled in the art.

In one embodiment, the oral dosage form comprises predefined release profile. In one embodiment, the oral dosage form of the present invention comprises an extended release tablets, capsules, lozenges or chewable tablets. In one embodiment, the oral dosage form of the present invention comprises a slow release tablets, capsules, lozenges or chewable tablets. In one embodiment, the oral dosage form of the present invention comprises an immediate release tablets, capsules, lozenges or chewable tablets. In one embodiment, the oral dosage form is formulated according to the desired release profile of the pharmaceutical active ingredient as known to one skilled in the art.

Peroral compositions, in some embodiments, comprise liquid solutions, emulsions, suspensions, and the like. In some embodiments, pharmaceutically-acceptable carriers suitable for preparation of such compositions are well known in the art. In some embodiments, liquid oral compositions comprise from about 0.012% to about 0.933% of the desired compound or compounds, or in another embodiment, from about 0.033% to about 0.7%.

In some embodiments, compositions for use in the methods of this invention comprise solutions or emulsions, which in some embodiments are aqueous solutions or emulsions comprising a safe and effective amount of the compounds of the present invention and optionally, other compounds, intended for topical intranasal administration. In some embodiments, h compositions comprise from about 0.01% to about 10.0% w/v of a subject compound, more preferably from about 0.1% to about 2.0, which is used for systemic delivery of the compounds by the intranasal route.

In another embodiment, the pharmaceutical compositions are administered by intravenous, intra-arterial, or intramuscular injection of a liquid preparation. In some embodiments, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, the compounds of the present invention are combined with an additional appropriate therapeutic agent or agents, prepared and applied as solutions, suspensions, or emulsions in a physiologically acceptable diluent with or without a pharmaceutical carrier.

In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

In one embodiment, pharmaceutical compositions for use in accordance with the present invention is formulated in conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

In one embodiment, injectables, of the invention are formulated in aqueous solutions. In one embodiment, injectables, of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, pharmaceutical compositions for parenteral administration include aqueous solutions of the active preparation in water-soluble form. Additionally, suspensions of the active ingredients, in some embodiments, are prepared as appropriate oily or water based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also contain suitable stabilizers or agents which increase the solubility of the active ingredients to allow for the preparation of highly concentrated solutions.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In another embodiment, the pharmaceutical composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref Biomed. Eng. 14:201 (1987); Buchwald et al., Surgery 88:507 (1980); Saudek et al., N Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In some embodiments, the active ingredient is in powder form for constitution with a suitable vehicle, e.g., sterile, pyrogen-free water based solution, before use. Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the active ingredients are contained in an amount effective to achieve the intended purpose. In some embodiments, a therapeutically effective amount means an amount of active ingredients effective to prevent, alleviate or ameliorate symptoms of disease or prolong the survival of the subject being treated.

In one embodiment, determination of a therapeutically effective amount is well within the capability of those skilled in the art.

The compositions also comprise preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise local anesthetics or other actives. The compositions can be used as sprays, mists, drops, and the like.

The compositions also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In some embodiments, compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. In another embodiment, the modified compounds exhibit substantially longer half-lives in blood following intravenous injection than do the corresponding unmodified compounds. In one embodiment, modifications also increase the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

In one embodiment, compositions of the present invention are presented in a pack or dispenser device, such as an FDA approved kit, which contain one or more unit dosage forms containing the active ingredient. In one embodiment, the pack, for example, comprise metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-Ill Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Animal Handling

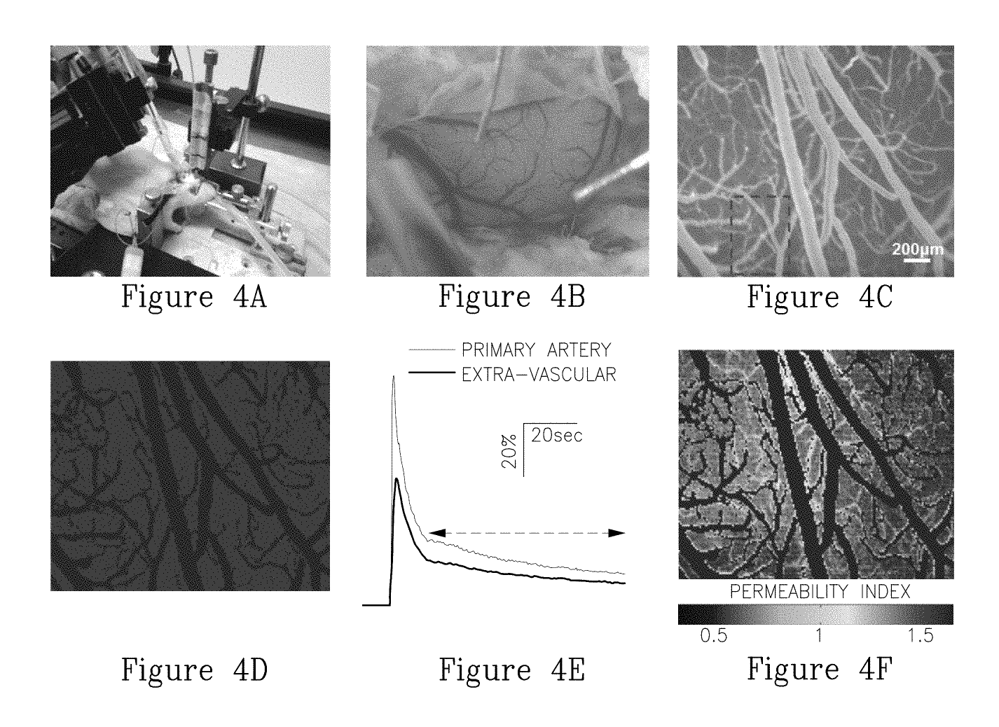

All experimental procedures in animals were approved by the Ben-Gurion University ethics committee for animal testing. Unless otherwise mentioned, all materials were purchased from Sigma-Aldrich ltd. Surgical procedures in male Spraugue-Dawley rats (200-380 g BW) were performed as previously reported (Prager et al., 2010). Rats were deeply anesthetized by intraperitoneal administration of either isoflurane or ketamine (100 mg/ml, 0.08 ml/100 gr) and xylasine (20 mg/ml, 0.06 ml/100 gr). The tail vein was catheterized, and animals were placed in a stereotactic frame (FIG. 4A) under a SteREO Lumar V12 fluorescence microscope (Zeiss, Gottingen, Germany).

Body temperature was continuously monitored and kept stable at 37±0.5° C. using a feedback-controlled heating pad (Physitemp Ltd.). Heart rate, breath rate and oxygen saturation levels were continuously monitored using STARR (Life Sciences Ltd.). A cranial section (4 mm caudal, 2 mm frontal, 5 mm lateral to bregma) was removed over the right sensory-motor cortex. The dura and arachnoid layers were removed (FIG. 4B) and the exposed cortex was continuously perfused with ACSF (Prager et al., 2010) containing (in mM): 124 NaCl, 26 NaHCO₃, 1.25 NaH₂PO₄, MgSO₄, 2 CaCl₂, 3 KCl, and 10 Glucose (pH 7.4).

To block neuronal activity tetrodotoxin (TTX) (Narahashi et al., 1964) (10 μM), 6-Cyano-2,3-dihydroxy-7-nitro-quinoxaline (CNQX) (Yoshiyama et al., 1995) (50 μM), D-AP5 (Morris, 1989) (50 μM) and picrotoxin (PTX) (Yoon et al., 1993) (50 μM) were added to the perfusing solution (ACSF). To induce prolonged seizures (status epilepticus, SE), 4AP (Uva et al., 2013) (500 μM) or PTX (100 μM) were added to the ACSF. Electrocorticgram (ECoG) was recorded using bi-polar electrodes and a telemetric recording system (DSI Ltd.). In some cases, thrombotic stroke was induced using photothrombosis (Prado, 1987). Detection of calcium ions in the rat cortex was done with calcium chelators as previously reported (Stosiek et al., 2003). 50 μg of the fluorescent chelator OGB were dissolved in 4 μl of dimethyl sulfoxide) DMSO) containing 20% pluronic f-127. This mixture was diluted in 36₁11 of a loading solution containing (in mM): 150 NaCl, 2.5 KCl, 10 HEPES, in ddH₂O (pH=7.4).

The exposed cortex was incubated with the final mixture for 1 hour. Probe-based confocal laser endomicroscopy (PCLE) was performed using Cellvizio dual band (Mauna Kea Technologies Ltd.) at 488 and 660 nm. Dynamic imaging of rCBF and BBB permeability measurements were performed as reported (Prager et al., 2010).

Additionally, qualitative assessment of BBB permeability was done by intravenously injecting the albumin-binding dye, EB (Wolman et al., 1981) (2.4 ml/kg, in 0.9% NaCl), extraction of brains following cardiac perfusion (4% paraformaldehyde in phosphate buffered saline) and verifying extravasation of the dye. Assessments of vascular diameter in the exposed cortical section were performed by transforming vascular pixel quantification into metric measurements.

### Fluorescent Angiography and BBB Permeability Assessment

Dynamic imaging of regional cerebral blood flow (rCBF) and BBB permeability measurements were performed as reported (Chassidim et al., 2014; Prager et al., 2010). The non-BBB permeable fluorescent dye, sodium fluorescein (NaFlu) was injected intravenously (1 mg/ml, 0.2 ml/injection, in 0.9% NaCl). Full-resolution (658×496 pixel) images of cortical surface vessels were obtained (5 frames/second, EMCCD camera: Andor Technology, DL-658 M-TIL, FIG. 4C) before, during and after injection of the tracer. Offline image analysis was carried out using MATLAB (MathWorks, Natick, USA) and included: sub-pixel image registration, segmentation using noise filtration, hole-filling and adaptive threshold to produce a binary image, separating blood vessels from extra-vascular regions (FIG. 4D). Signal intensity changes over time and space were then analyzed so that each pixel was represented by intensity vs. time (IT) curve (FIG. 4E). An arterial IT curve (AIF) was created by spatially averaging signal intensity through time in the primary artery. A BBB permeability index (PI) (FIG. 4E) was calculated for each extra-vascular pixel as the ratio between IT curve and AIF, from the point of the second decline phase to the end of the measurement

$$( \text{∼} 250 - 300 s ) \text{:} P I = \frac{1}{T} {\int }_{{t}_{cr}}^{{t}_{end}} \frac{{I}_{EV}}{{I}_{AIF}} ( t ) dt T = {t}_{end} - {t}_{cr} .$$

The PI indicates how much tracer remains in extra-vascular tissue in relation to the applied amount. PI>1 indicates tracer accumulation and defines BBB dysfunction. Fitting a PI to each extra-vascular pixel enabled spatial mapping of BBB permeability (FIG. 4F). The PI for each vascular pixel was set to 0, and therefore vessels were excluded from the map. The global permeability of a region was calculated by averaging its PI values. Permeability measurements with this approach are possible only for regions with fully functional vasculature as the transfer of molecules between vessel and parenchyma is quantified. Increased permeability in damaged vessels is not depicted here. The method was validated in well-established models of BBB dysfunction such as cortical perfusion of sodium deoxycholate and photo-induced stroke (Chassidim et al., 2014; Prager et al., 2010; Schoknecht et al., 2014).

### Thrombotic Stroke

The photo-reactive substance, rose bengal (RB) (7.5 mg/ml, 0.133 ml/100 g), was injected into the tail vein while a vascular region in the exposed cortex was laser-illuminated at 523 nm (CNI lasers, 5 mW). The transformation of RB into free radicals, binding to platelets and the formation of clots occurred within 15 minutes (Prager et al., 2010; Schoknecht et al., 2014).

### Electrocorticogram Recording and Analysis

ECoG was recorded using a telemetric system (DSI Ltd.). Two electrodes were implanted, one attached to an intra-cranial screw adjacent to the exposed cortex, and the second placed over the exposed cortex while secured with bone wax and dental cement. In-house MATLAB scripts were used to display and record signals for post-processing. Signals were sampled at 200 Hz and filtered using a MATLAB simulated Butterworth filter, so to display only the desired frequency band (10-40 Hz). The mean power was calculated using the MATLAB “pwelch” function.

### Magnetic Resonance Imaging for BBB Permeability Assessment in Humans

The human trial was approved by the local ethics committee at La-Sapienza University, Rome. All patients gave written consent for participation in the trial. A total of nineteen subjects (ages 32-76, ten males) with histologically confirmed GBM (grade IV) were enrolled for a short pilot study. In addition four non-tumor control subjects were recruited. Magnetic resonance imaging (MM) was performed with a 1.5T Intera scanner (Phillips) containing a six-element receiver coil. A standard battery of anatomical scans was performed. These scans included diffusion-weighted imaging, fluid-attenuated inversion recovery (FLAIR), T2-weighted scans, as well as a high-resolution T1-weighted anatomical scan (3-D gradient echo, TR/TE/TI: 8.6/3.5/900 ms, FOV: 240×240 mm, matrix:

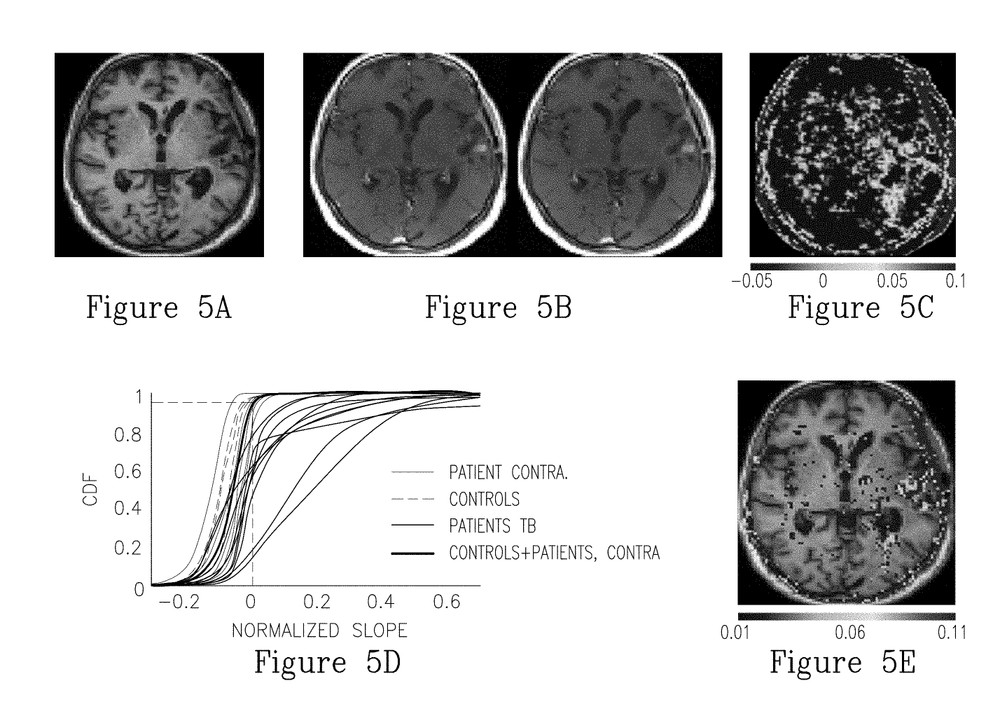

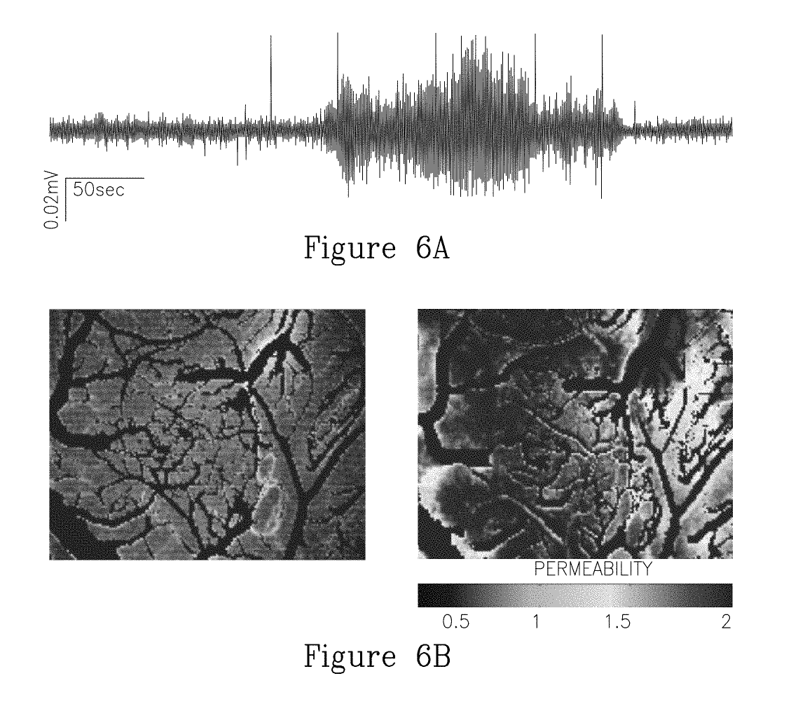

256×256, slice thickness: 1.2 mm, 150 slices, flip angle: 8°). In addition, two baseline scans were performed with dynamic contrast enhanced MM (DCE-MRI) (spin echo, TR/TE: 1000/8 ms, FOV: 240×180 mm, matrix: 256×192, slice thickness: 3 mm with no gap, forty four slices, two concatenations, acquisition time: 3 minutes, 14 seconds). The DCE-MRI acquisition consisted of at least seven longitudinal scans using the same protocol (FIG. 5A-B).

Images were processed using Statistical Parametric Mapping (SPM, http://www.fil.ion.ucl.ac.uk/spm), FSL (FMRIB, UK), ImageJ (NIH, USA) as well as with in-house scripts created with MATLAB. Pre-processing was carried out using SPM and included co-registration, segmentation, spatial normalization and Gaussian smoothing with a 2×2×6 (x y z) mm kernel. A simplified form of DCE-MRI analysis was employed as previously published (Chassidim et al., 2013). A linear curve was fitted to the dynamic data (seven time points, 3-21 minutes following entry to the scanner), generating a slope value for each voxel (“slope map”, FIG. 5C). A negative slope indicated normal washout of the contrast agent from the vascular compartment, while a positive slope suggested accumulation of the contrast agent in areas with absent or compromised BBB. Method validation was achieved by demonstration of: (1) Positive slope measured in tissues lacking BBB (e.g. extra-cranial muscle); (2) Positive slope measured in areas of tumors and surrounding tissue; (3) Negative slope measured in major blood vessels such as the venous sinuses. To enable intra- and inter-subject comparisons and compensate for potential differences in injection and blood flow slope maps were normalized by dividing each voxel by the mean slope value in a region of interest (ROI) drawn in the superior sagittal sinus: slₜ=slₜ/sl_{sag} (sl: slope, t: tissue, sag: superior sagittal sinus, respectively (Chassidim et al., 2013)). Subsequent analysis was restricted to voxels assigned as grey or white matter components from segmentation of the high-resolution anatomical scan. The upper limit of normal vascular permeability was calculated using the cumulative distribution function (CDF) of the tissue-masked slope maps (FIG. 5D). These were derived from the slope values of the hemisphere contralateral to the resected tumor and both hemispheres for the control group, in both cases following “sham” brain stimulation. The 95^{th} percentile of the combined CDF was defined as the threshold (FIG. 5D). The following masks of anatomical areas of interest were created and used to measure permeability values: tumor bed, peritumoral area, contralateral hemisphere relative to the tumor and ipsilateral hemisphere relative to the tumor (the latter region excluding the tumor bed, see FIG. 5C).

Note that the term “tumor bed” is used interchangeably with the region corresponding to the resected tumor zone. The mask of the tumor bed was created by tracing the resected tumor outline on each slice of both the high resolution anatomical scan and the first DCE-MRI scan following sham stimulation. The conjunction of these two masks was then defined as the tumor bed. The peritumoral region was created by subtraction of the tumor bed mask from a dilated tumor bed mask (created in FSL by mean dilation of non-zero voxels). For each mask of every scan, two parameters were calculated: (1) The mean value of the slope map, and (2) The percentage of voxels with abnormally high slope values (suprathreshold-ST). Examination of the effect on BBB permeability was performed using these two parameters. Slope differences were calculated as: 100×(sl₁−sl₂)/|sl₂|.

### Statistical Analysis

Unless otherwise mentioned, mean±square error of the mean (SEM) are described. All comparisons were made using two-tailed Mann-Whitney-U or Wilcoxon Ranked Sum tests (Mann-Whitney or Wilcoxon respectively, see text). P=0.05 was defined as the level of significance. Statistical analysis was performed using SPSS (IBM, Armonk, USA).

### Example 1: Seizures Result in BBB Opening

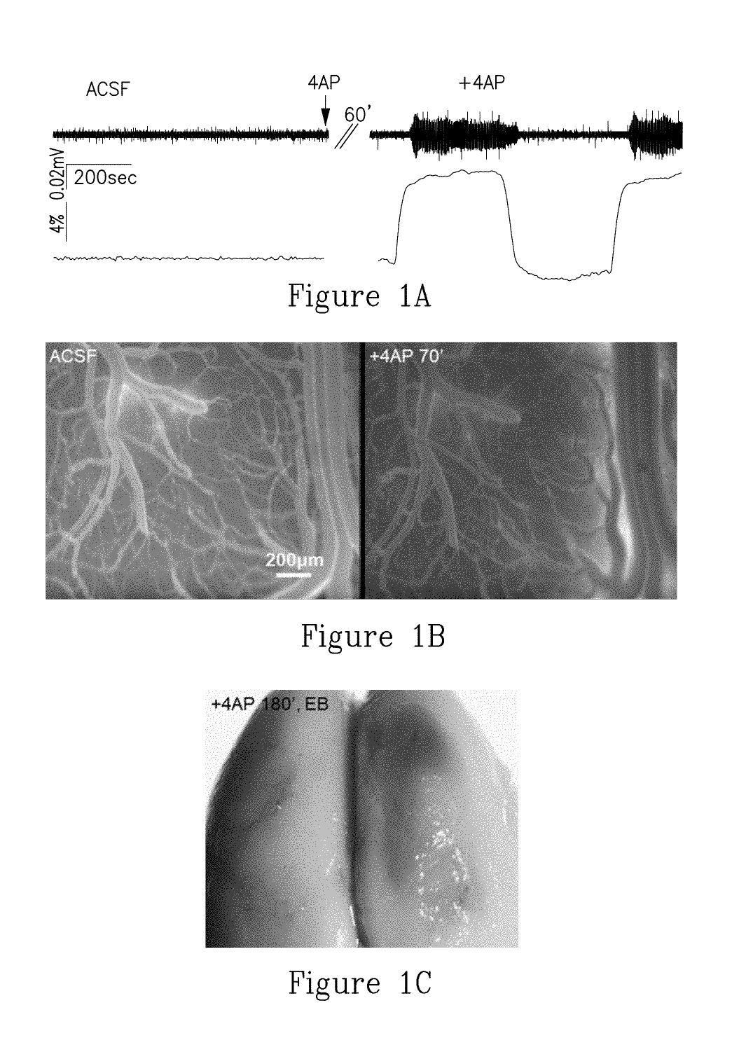

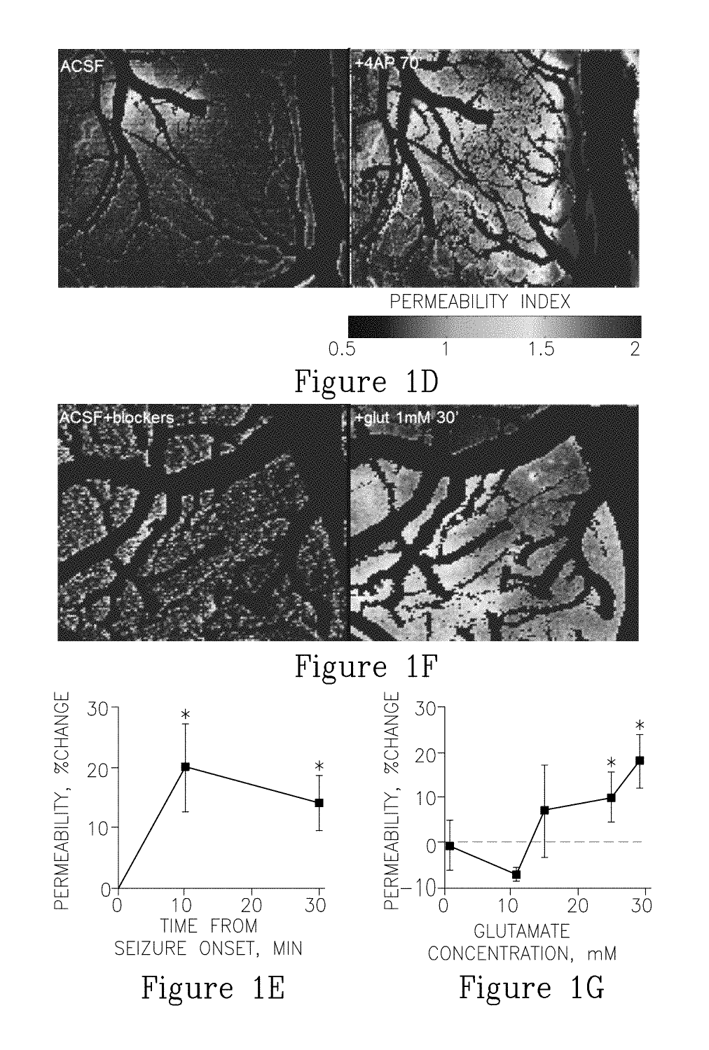

First it was tested whether focally-induced cortical seizures are associated with increased vascular permeability. Using intravital microscopy and the open-window method (FIG. 4, see Materials and Methods) for parallel vascular imaging and ECoG recordings (FIG. 1A), we induced recurrent seizures using either the potassium channel blocker 4AP, or PTX, blocker of the gamma-aminobutyric acid A (GABA-A) receptor (n=6 and n=2, respectively). We quantitatively assessed BBB integrity by analyzing angiographic fluorescence imaging data (Prager et al., 2010) (FIG. 4). Seizures were accompanied by a significant immediate increase in vessel diameter (10.05±1.01%, n=8, P=0.01, Wilcoxon, FIG. 1A). Vessel permeability to NaFlu increased as soon as ˜10 minutes from seizure onset (20.01±7.24%, n=8, P=0.01, Wilcoxon, FIG. 1B/D-E) and remained elevated during recurrent seizures (30 minutes from seizure onset permeability increased by 14.17±4.65%, n=7, P=0.02, Wilcoxon, FIG. 1E).

### Example 2: Excessive Glutamate Release Enhances Vascular Permeability

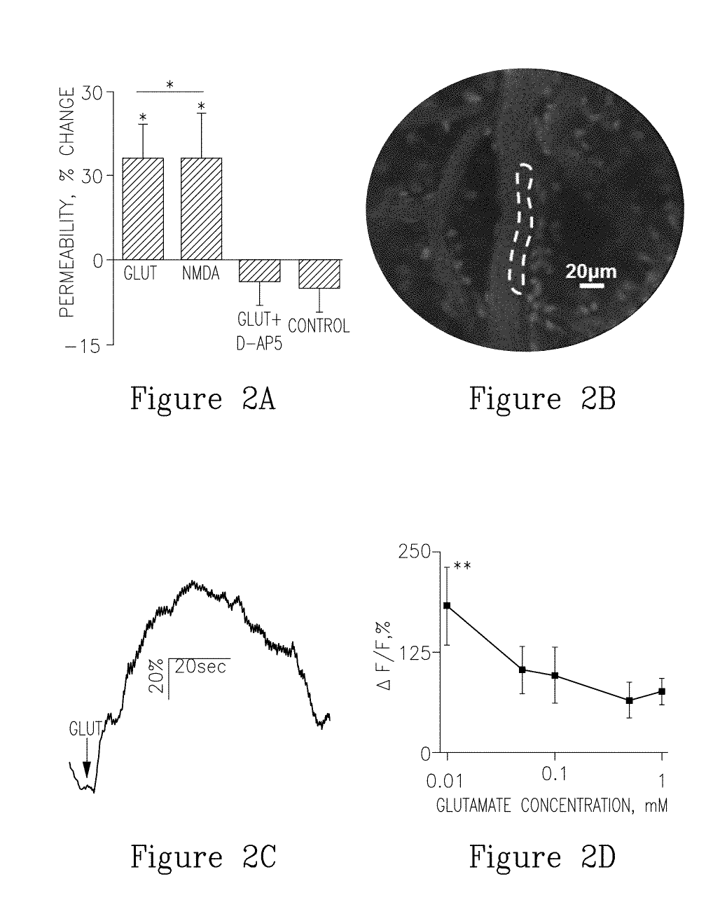

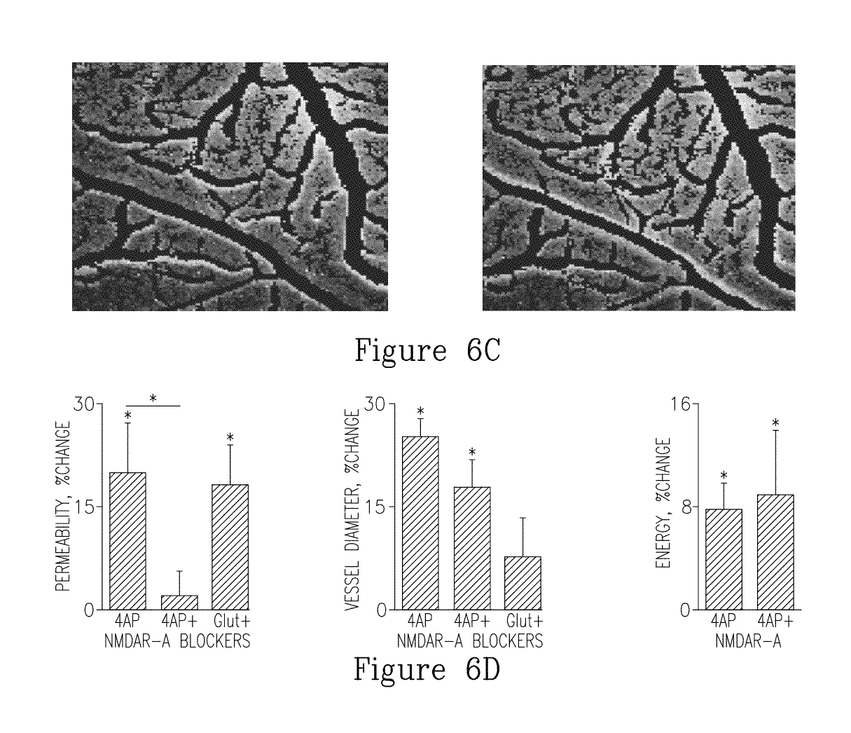

Hyper-synchronization and activation of large neuronal populations is associated with a massive release of the excitatory neurotransmitter glutamate (Bradford, 1995). In cultured brain endothelial cells, the expression of glutamate receptors has been reported (András et al., 2007; Krizbai et al., 1998; Sharp et al., 2003), and exposure to glutamate (1 mM) resulted in NMDA-R mediated reduction in the levels and cellular redistribution of the tight junction protein occludin, as well as in lower electrical resistance (András et al., 2007; Sharp et al., 2003). To test the hypothesis that excess glutamate mediates BBB dysfunction in vivo, we directly perfused the cortex of rats with increasing concentrations of glutamate (0.01-1 mM). To exclude indirect effects of glutamate via neuronal activation, we blocked neuronal firing, main excitatory and inhibitory GABAergic synaptic transmission using TTX, CNQX and PTX, respectively. ECoG was recorded simultaneously to confirm reduction in neuronal activity and to exclude the induction of seizures under these experimental conditions (data not shown). Local exposure of the neocortex to glutamate increased vessel permeability in a dose-dependent manner that reached significance at 1 mM (18.15%±5.9%, n=9, P=0.02, Wilcoxon, FIG. 1F-G, FIG. 2A). Glutamate application was not accompanied by a significant change in vessel diameter (3.12±2.24%, P=0.16, Wilcoxon, FIG. 6D).

To test whether the increase in endothelial permeability was attributed to NMDA-R, experiments were repeated with cortical perfusion of NMDA (1 mM) and with glutamate in the presence of the NMDA-R antagonist D-AP5 (50 μM). While NMDA, similar to glutamate, increased vessel permeability (18.44±7.58%, n=5, P=0.04, Wilcoxon, FIG. 2A), in the presence of D-AP5 glutamate had no effect on vessel permeability (−3.81±4.34%, n=5, P=0.22, Wilcoxon, FIG. 2A)

In control experiments, brains were exposed to ACSF for 60-120 min, no significant change in permeability was measured, excluding a time-dependent increase in permeability (“control”, −5.11±3.07%, n=16, P=0.7, Wilcoxon, FIG. 2A). Since NMDA-R conduct calcium ions (De Bock et al., 2013), we used the calcium-sensitive fluorophore OGB (FIG. 2B, see Materials and Methods) to follow changes in calcium levels in vessels' wall in response to drop application of glutamate (0.01-1 mM). We confirmed a long-lasting (12.5±1.3 sec, n=109) increase in endothelial intracellular calcium following a drop application of glutamate (n=4, P<0.01, Wilcoxon, FIG. 2C-D). These findings suggest that even in the absence of neuronal firing, exposure of brain microvasculature to glutamate results in NMDA-R-mediated increase in intracellular calcium and permeability.

### Example 3: Therapeutic Implications

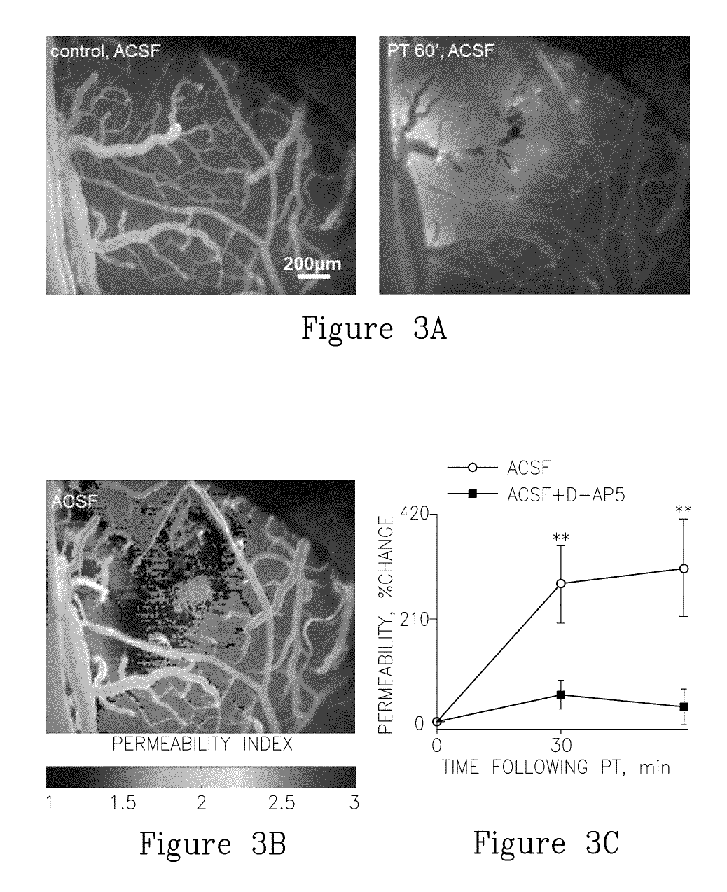

Since excess glutamate release is a hallmark of brain hypoxic-ischemic injuries (Benveniste et al., 1984; Nishizawa, 2001; Rothman and Olney, 1986), status epilepticus and brain trauma and since the dynamic progression of BBB dysfunction has been characterized in the rat cerebral cortex stroke photothrombosis model (Schoknecht et al., 2014), we tested the hypothesis that blocking NMDA-R activation could reduce BBB breakdown in the peri-ischemic brain. A focal ischemic lesion was induced by photothrombosis following an injection of RB (Watson et al., 1987; Prager et al., 2010; Schoknecht et al., 2014) (FIG. 3A) in the presence or absence of the specific NMDA-R blocker, D-AP5. The spatial progression of BBB dysfunction in the peri-ischemic region was significantly reduced in the presence of D-AP5 at 30 and 60 minutes after clot induction (P=0.03, Mann-Whitney, FIG. 3B-C).

The BBB is the hallmark of normal brain vascularization, enabling the unique extracellular neuronal environment essential for its proper function. Vascular pathology and dysfunctional BBB are common in brain diseases, particularly described in ischemic and traumatic brain injuries (Prager et al., 2010; Schoknecht et al., 2014) but also in aging and neurodegenerative disorders (Mecocci et al., 1991; Montagne et al., 2015), as well as in peripheral diseases affecting the brain (Mooradian, 1997) (e.g. hypertension, diabetes mellitus). Increased endothelial permeability to serum proteins has been found to induce an astrocytic transformation associated with neuroinflammation and impaired control of the extracellular milieu, neuronal hyperexcitability, synaptogenesis and pathological plasticity (Cacheaux et al., 2009; David et al., 2009; Weissberg et al., 2015). Experimental and clinical evidence thus support the notion that a compromised BBB may be associated with dysfunction of the neurovascular network, cognitive and emotional impairments (Montagne et al., 2015), seizures and epilepsy (Friedman, 2011) as well as neurodegeneration (Zlokovic, 2008), thus highlighting vascular integrity as a target for treatment. However, there is lack of knowledge regarding the mechanisms of BBB opening under disease conditions and no therapeutics available to modulate BBB integrity. For decades, the intact BBB, as a major obstacle for drug delivery into the brain, has been the target of research and clinical trials aiming to transiently increase its permeability. In the present study we show that high concentrations of glutamate, the major excitatory brain neurotransmitter, directly modulate vascular permeability. The current results demonstrated that glutamate enhances calcium influx into brain endothelial cells and facilitates their permeability through activation of NMDA-R. We demonstrate novel therapeutic implications of our findings: that increased BBB permeability in the peri-ischemic brain (a common finding in stroke patients and a risk factor for hemorrhagic complication in the presence of antithrombotic treatment) and following seizures can be prevented using NMDA-R antagonists.

It was shown that the induction of seizures in vivo is associated with an increase in vessel diameter and permeability to both low and high molecular weight substances (FIG. 1). Prolonged or frequently recurring seizures, as well as ischemic stroke and traumatic brain injury, are associated with increased concentrations of extracellular glutamate (over 50 fold increase, and in the 0.1-1 mM range).

The current data suggest that neuronal release of glutamate is targeting a non-neuronal target. Interestingly, in the absence of neuronal activity glutamate did not induce an increase in vessel diameter, suggesting different mechanisms underlying the coupling between neuronal activity to vascular diameter response and permeability. The observations that direct application of NMDA similarly enhances permeability, and that permeability increase is blocked in the presence of NMDA-R blockers (FIG. 2A) indicate that the effect of glutamate is mediated by NMDA-R. Our experiments showing increased intracellular calcium in vascular endothelium in response to glutamate are consistent with previous in vitro data showing that NMDA-mediated effects are dependent on increased intracellular calcium (Krizbai et al., 1998; Sharp et al., 2003). However, we cannot rule out non-calcium dependent signaling mechanisms and the role of other components of the neurovascular unit, such as astrocytes and pericytes, which could also respond to glutamate and signal brain endothelial cells to alter permeability (Carmignoto and Gómez-Gonzalo, 2010). Preventing intracellular calcium elevation using calcium chelators (e.g. BAPTA-AM), is not feasible in vivo due to massive vasoconstriction and associated ischemia (data not shown). The intracellular signaling leading to enhanced permeability is also not clear. While some studies suggest tight junction reorganization (De Bock et al., 2013), others suggest the down-regulation of tight junction elements (András et al., 2007). The latter seems unlikely with the relatively short time delay between insult (seizure/stroke) and enhanced permeability. Modulation of transcellular transport mechanisms (e.g. intercellular adhesion molecule-ICAM mediated) (Yang et al., 2005)) may also be involved.

Brain insults, including hypoxic-ischemic or traumatic injuries, were shown to be associated with synchronous neuronal hyper-excitability and elevated extracellular glutamate in both animal models and man (Benveniste et al., 1984; Nishizawa, 2001; Rothman and Olney, 1986). Interestingly, while NMDA-R antagonists were consistently shown to be neuroprotective in animal models of brain injuries (Huang and Wang, 2014; Miguel-Hidalgo et al., 2002; Rao et al., 2001), their effects on brain vasculature have never been carefully tested. We propose that at least part of the protecting effect of NMDA-R blockers may be due to their therapeutic effect to reduce BBB breakdown within the peri-ischemic/peri-injured brain. The failure of NMDA-R antagonists as neuroprotectants in clinical trials (Ikonomidou and Turski, 2002; Morris et al., 1999) might thus be due to variability between patients in the extent of BBB damage (Friedman et al., 2014). This testable hypothesis calls for BBB imaging in patients with brain insults and follow-up of NMDA-R effects on vascular integrity and clinical outcome in specific sub groups of patients.

In summary, we present a novel neuronal-activity mediated, NMDA-R dependent mechanism for the modulation of brain vasculature's permeability. We propose that this mechanism may be exploited for facilitating BBB closure in neurological disorders and opening in tumor patients to enhance drug delivery.

### Example 4: NMDAR Antagonists Diminish BBB Opening in Rat Models of Brain Injury and are Associated with Better Outcome

It was hypothesized, that treatment of brain disorders with antagonists to glutamate receptors can reduce BBB opening and associated brain damage. This study combined both local application of the NMDA-R antagonist, D-AP5, as well as systemic administration of the FDA approved NMDA-R antagonist, memantine.

The present results suggest that the NMDAR antagonist D-AP5 significantly diminishes BBB dysfunction in the peri-ischemic region following photothrombosis (FIG. 3).

This experiment further provides that NMDAR antagonists' protective effect is in large part due to their effect on brain vasculature, specifically facilitating BBB closure, and thus should be indicated only to those patients with a BBB pathology.

Since prolonged and recurrent epileptic seizures may also be associated with vascular pathology and damage to the BBB through the activation of NMDA-R, the effect of NMDA-R antagonists after the induction of recurrent seizures in anesthetized rats was tested. The epileptogenic substance 4AP was applied on the surface of the brain while monitoring ECoG.

Animals were treated with either topical application of the specific NMDA-R blocker D-AP5 (100 μM) (prior to and during seizure induction), or systemically with the FDA approved memantine (40 mg/kg, intraperitonealy, immediately following seizure onset). ECoG recordings verified no impact of the treatment on seizure intensity (FIG. 6 A/D).

However, treatment with NMDA-R antagonism diminished BBB damage significantly (FIG. 6B-D, P=0.02, n=6 Vs. n=8, Mann-Whitney) without blocking the normal vasodilating response to neuronal hyperactivity (FIG. 6D).

1. A method for reducing the permeability of the blood-brain-barrier, in a subject afflicted with a brain injury, comprising administering to said subject, a composition comprising N-methyl-d-aspartate receptor (NMDA-R) antagonist, thereby reducing the permeability of the blood-brain-barrier, in a subject afflicted with a brain injury.

2. The method of claim 1, wherein said NMDA-R antagonist is a competitive antagonist.

3. The method of claim 2, wherein said NMDA-R competitive antagonist is APV, R-2-amino-5-phosphonopentanoate (AP5), memantine, 2-amino-7-phosphonoheptanoic acid (AP7), 3-[(R)-2-carboxypiperazin-4-yl]-prop-2-enyl-1-phosphonic acid (CPPene), (2S,4R)-4-(phosphonomethyl)piperidine-2-carboxylic acid (Selfotel) or any combination thereof.

4. The method of claim 1, wherein said subject is afflicted with brain seizures, status epilepticus, BBB pathology, or a combination thereof.

5. (canceled)

6. The method of claim 1, wherein said subject is afflicted with brain ischemia.

7. The method of claim 1, wherein said reducing the permeability is transiently reducing the permeability.

8. A method for preventing an increase in the permeability of the blood-brain-barrier, in a subject at risk of a brain injury, comprising administering to said subject, a composition comprising N-methyl-d-aspartate receptor (NMDA-R) antagonist, thereby preventing an increase in the permeability of the blood-brain-barrier, in a subject at risk of a brain injury.

9. The method of claim 8, wherein said NMDA-R antagonist is a competitive antagonist.

10. The method of claim 9, wherein said NMDA-R competitive antagonist is APV, R-2-amino-5-phosphonopentanoate (AP5), 2-amino-7-phosphonoheptanoic acid (AP7), 3-[(R)-2-carboxypiperazin-4-yl]-prop-2-enyl-1-phosphonic acid (CPPene), (2S,4R)-4-(phosphonomethyl)piperidine-2-carboxylic acid (Selfotel) or any combination thereof.

11. (canceled)

12. The method of claim 8, wherein said subject is afflicted with brain seizures, status epilepticus, brain injury, BBB pathology, or a combination thereof.

13. The method of claim 12, wherein said brain injury is traumatic brain injury (TBI).

14. The method of claim 8, wherein said preventing an increase in the permeability is transiently preventing an increase in the permeability.
